(19) United States
(12) Patent Application Publication
(10) Pub. No.: US 2011/0178180 A1
(43) Pub. Date: Jul. 21, 2011

(54) DEUTERIUM-ENRICHED COLCHICINE, THIOCOLCHICINE, AND DERIVATIVES THEREOF; METHODS OF PREPARATION; AND USE THEREOF

(76) Inventors: Kurt Nielsen, Chadds Ford, PA (US); Rajesh Manchanda, Sudbury, MA (US)

(21) Appl. No.: 13/007,851

(22) Filed: Jan. 17, 2011

Related U.S. Application Data
(60) Provisional application No. 61/295,818, filed on Jan. 18, 2010.

(51) Int. Cl. A61K 31/165; C07C 233/01; A61P 29/00; A61P 21/02; A61P 19/06

(52) U.S. Cl. 514/629; 564/222

(57) ABSTRACT

Disclosed herein are deuterium-enriched colchicine, thiocolchicine, and derivatives thereof. The deuterium-enriched compounds are useful as, an antiproliferative agent, a muscle relaxant, an anti-inflammatory agent, or an anti-gout agent.

### CROSS REFERENCE TO RELATED APPLICATION

This application claims the benefit of U.S. Provisional Application Ser. No. 61/295,818 filed Jan. 18, 2010, which is hereby incorporated by reference in its entirety.

### BACKGROUND

Thiocolchicine and colchicine are known semisynthetic and natural alkaloids, respectively. Thiocolchicine is an inhibitor of microtubules by specific binding to tubulin. Colchicine is a known gout suppressant and agent for the treatment of Familial Mediterranean Fever.

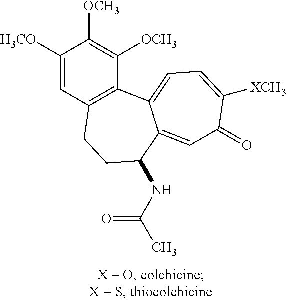

The thiocolchicine derivative thiocolchicoside (N-[(7S)-3-(β-D-glucopyranosyloxy)-5,6,7,9-tetrahydro-1,2-dimethoxy-10-(methylthio)-9-oxobenzo[a]heptalen-7-yl-]-acetamide also known as 3-demethylthiocolchicine glucoside; CAS Registry No. 602-41-5) is a known skeletal muscle relaxant. Studies have suggested that thiocolchicoside is metabolized in vivo into an aglycone derivative via deglycosylation and subsequent formation of a 3-O-glucuronidated aglycone derivative. See, Trellu et al., “New metabolic and pharmacokinetic characteristics of thiocolchicoside and its active metabolite in healthy humans”, Fundamental & Clinical Pharmacology, 18, (2004) 493-501. The aglycone derivative exhibited no muscle relaxant activity in a rat model while the 3-O-glucuronidated aglycone derivative was found to exhibit muscle relaxant activity similar to that of thiocolchicoside. Id.

There remains a need in the art for new compounds exhibiting muscle relaxant activity, anti-gout activity, or other therapeutic benefits having greater safety profile, activity, or therapeutic index than thiocolchicoside, thiocolchicine, or colchicine.

### SUMMARY

The above-described and other drawbacks are alleviated by a deuterium-enriched compound of Formula I,

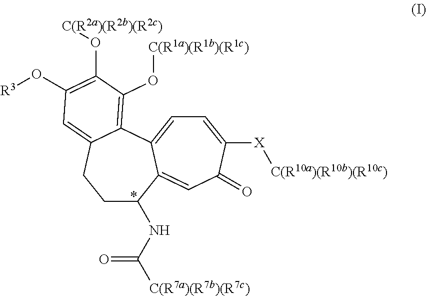

or a pharmaceutically acceptable salt, hydrate, or solvate thereof, wherein:

### X is —O— or —S—;

R^{1a}, R^{1b}, R^{1c}, R^{2a}, R^{2b}, R^{2c}, R^{3m}, R^{7a}, R^{7b}, R^{7c}, R^{10a}, R^{10b}, and R^{10c}, are each independently hydrogen or deuterium; and

### R³ is

R^{3m}, wherein R^{3m} is hydrogen, deuterium, or C(R^{3a})(R^{3h})(R^{3e}) wherein R^{3a}, R^{3h}, R^{3e}, are each independently hydrogen or deuterium, or

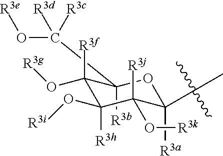

wherein R^{3a}, R^{3h}, R^{3e}, R^{3d}, R^{3e}, R^{3f}, R^{3g}, R^{3h}, R³ⁱ, R^{3j}, R^{3k} are each independently hydrogen or deuterium, or

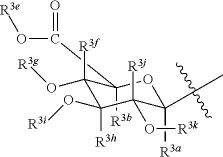

wherein R^{3a}, R^{3b}, R^{3c}, R^{3f}, R^{3g}, R^{3h}, R³ⁱ, R^{3j}, R^{3k} are each independently hydrogen or deuterium; and
the stereocenter of Formula I indicated by “*” can be racemic, a mixture of R and S enriched in either the R or S isomer, in the R configuration, or in the S configuration;
provided that
when R³ is R^{3m}, the abundance of deuterium in at least one of R^{1a}, R^{1b}, R^{1c}, R^{2a}, R^{2b}, R^{2c}, R^{3a}, R^{3b}, R^{3c}, R^{3m}, R^{7a}, R^{7c}, R^{10c}, R^{10b}, and R^{10c} is at least 3, 5, 10, 20, 40, 60, 80, 90, 95, 98, or 99 mol %;
when R³ is

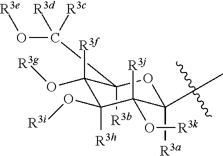

the abundance of deuterium in at least one of R^{1a}, R^{1b}, R^{1c}, R^{2a}, R^{2b}, R^{2c}, R^{3a}, R^{3b}, R^{3c}, R^{3d}, R^{3e}, R^{3f}, R^{3g}, R^{3h}, R³ⁱ, R^{3j}, R^{3k}, R^{7a}, R^{7b}, R^{7c}, R^{10a}, R^{10b}, and R^{10c} is at least 3, 5, 10, 20, 40, 60, 80, 90, 95, 98, or 99 mol %; and
   when R³ is

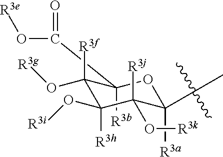

the abundance of deuterium in at least one of R^{1a}, R^{1b}, R^{1c}, R^{2a}, R^{2b}, R^{2c}, R^{3a}, R^{3b}, R^{3f}, R^{3g}, R^{3h}, R³ⁱ, R^{3j}, R^{3k}, R^{7a}, R^{7b}, R^{7c}, R^{10a}, R^{10b}, and R^{10c} is at least 3, 5, 10, 20, 40, 60, 80, 90, 95, 98, or 99 mol %.

In another embodiment, a deuterium-enriched compound of Formula II,

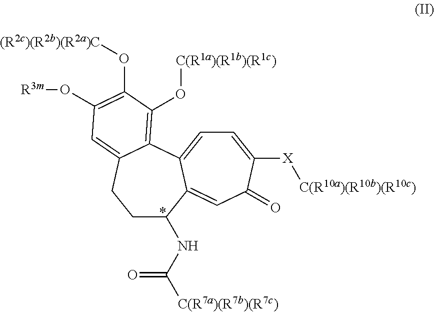

or a pharmaceutically acceptable salt, hydrate, or solvate thereof, wherein

### X is —O— or —S—;

R^{1a}, R^{1b}, R^{1c}, R^{2a}, R^{2b}, R^{2c}, R^{3m}, R^{7a}, R^{7b}, R^{7c}, R^{10a}, R^{10b}, and R^{10c} are each independently hydrogen or deuterium, provided that the abundance of deuterium in at least one of R^{1a}, R^{1b}, R^{1c}, R^{2a}, R^{2b}, R^{2c}, R^{3m}, R^{7a}, R^{7b}, R^{7c}, R^{10a}, R^{10b}, and R^{10c} is at least 3, 5, 10, 20, 40, 60, 80, 90, 95, 98, or 99 mol %; and
the stereocenter of Formula II indicated by “*” can be racemic, a mixture of R and S enriched in either the R or S isomer, in the R configuration, or in the S configuration.

In yet another embodiment, a deuterium-enriched compound of Formula III,

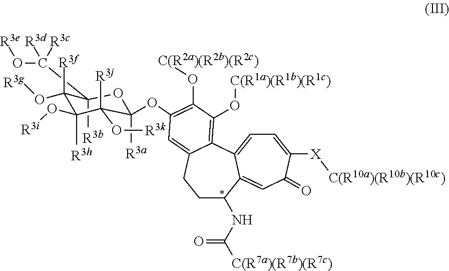

or a pharmaceutically acceptable salt, hydrate, or solvate thereof, wherein:

### X is —O— or —S—;

R^{1a}, R^{1b}, R^{1c}, R^{2a}, R^{2b}, R^{2c}, R^{3a}, R^{3b}, R^{3c}, R^{3d}, R^{3e}, R^{3f}, R^{3g}, R^{3h}, R³ⁱ, R^{3j}, R^{3k}, R^{7a}, R^{7b}, R^{7c}, R^{10a}, R^{10b}, and R^{10c}, are each independently hydrogen or deuterium, provided that the abundance of deuterium in at least one of R^{1a}, R^{1b}, R^{1c}, R^{2a}, R^{2b}, R^{2c}, R^{3a}, R^{3b}, R^{3c}, R^{3d}, R^{3e}, R^{3f}, R^{3g}, R^{3h}, R³ⁱ, R^{3j}, R^{3k}, R^{7a}, R^{7b}, R^{7c}, R^{10a}, R^{10b}, and R^{10c} is at least 3, 5, 10, 20, 40, 60, 80, 90, 95, 98, or 99 mol %; and
the stereocenter of Formula III indicated by “*” can be racemic, a mixture of R and S enriched in either the R or S isomer, in the R configuration, or in the S configuration.
provided that

at least one of R¹, R², R⁷, and R¹⁰ is —CH₂D, —CHD₂, or —CD₃; or

at least one of R^{3a}, R^{3b}, R^{3c}, R^{3d}, R^{3e}, R^{3f}, R^{3g}, R^{3h}, R³ⁱ, R^{3j}, R^{3k} is deuterium.

In another embodiment, a deuterium-enriched compound of Formula IV,

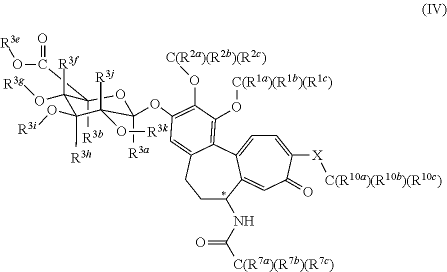

or a pharmaceutically acceptable salt, hydrate, or solvate thereof, wherein:

### X is —O— or —S—;

R^{1a}, R^{1b}, R^{1c}, R^{2a}, R^{2b}, R^{2c}, R^{3a}, R^{3b}, R^{3e}, R^{3f}, R^{3g}, R^{3h}, R³ⁱ, R^{3j}, R^{3k}, R^{7a}, R^{7b}, R^{7c}, R^{10a}, R^{10b}, and R^{10c} are each independently hydrogen or deuterium, provided that the abundance of deuterium in at least one of R^{1a}, R^{1b}, R^{1c}, R^{2a}, R^{2b}, R^{2c}, R^{3a}, R^{3b}, R^{3f}, R^{3g}, R^{3h}, R³ⁱ, R^{3j}, R^{3k}, R^{7a}, R^{7b}, R^{7c}, R^{10a}, R^{10b}, and R^{10c} is at least 3, 5, 10, 20, 40, 60, 80, 90, 95, 98, or 99 mol %; and

the stereocenter of Formula IV indicated by “*” can be racemic, a mixture of R and S enriched in either the R or S isomer, in the R configuration, or in the S configuration.

Also disclosed are pharmaceutical compositions comprising a deuterium-enriched compound, as well as methods of treating a patient in need thereof by administration of a deuterium-enriched compound or a composition comprising a deuterium-enriched compound to the patient.

### DETAILED DESCRIPTION

Disclosed herein are deuterium-enriched thiocolchicine, colchicine, and derivatives thereof (“deuterium-enriched compound”), methods of preparation thereof, compositions comprising a deuterium-enriched compound, and uses thereof as an active agent, particularly as a muscle relaxant, anti-gout agent, anti-proliferative agent, anti-cancer agent, or anti-inflammatory agent.

As used herein “deuterium-enriched compound” is a compound containing more than the natural abundance of deuterium, that is greater than 0.015 mol % deuterium.

In one embodiment, the deuterium-enriched compound is a compound of Formula I,

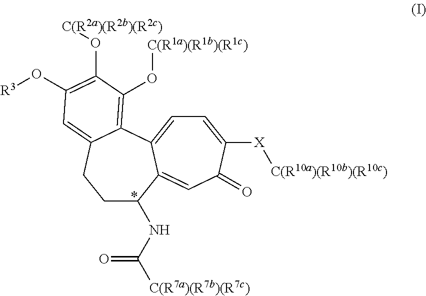

or a pharmaceutically acceptable salt, hydrate, or solvate thereof, wherein:

### X is —O— or —S—;

R^{1a}, R^{1b}, R^{1c}, R^{2a}, R^{2b}, R^{2c}, R^{3m}, R^{7a}, R^{7b}, R^{7c}, R^{10a}, R^{10b}, and R^{10c}, are each independently hydrogen or deuterium; and

### R³ is

R^{3m}, wherein R^{3m} is hydrogen, deuterium, or C(R^{3a})(R^{3b})(R^{3c}) wherein R^{3a}, R^{3b}, R^{3c}, are each independently hydrogen or deuterium, or

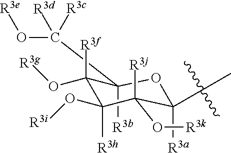

wherein R^{1a}, R^{3b}, R^{3c}, R^{3d}, R^{3e}, R^{3f}, R^{3g}, R^{3h}, R³ⁱ, R^{3j}, R^{3k} are each independently hydrogen or deuterium, or

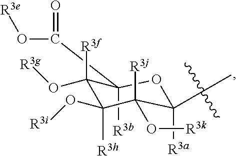

wherein R^{3a}, R^{3b}, R^{3e}, R^{3f}, R^{3g}, R^{3h}, R³ⁱ, R^{3j}, R^{3k} are each independently hydrogen or deuterium; and
the stereocenter of Formula I indicated by “*” can be racemic, a mixture of R and S enriched in either the R or S isomer, in the R configuration, or in the S configuration;
provided that when R³ is R^{3m}, the abundance of deuterium in at least one of R^{1a}, R^{1b}, R^{1c}, R^{2a}, R^{2b}, R^{2c}, R^{3a}, R^{3b}, R^{3c}, R^{3m}, R^{7a}, R^{7b}, R^{7c}, R^{10a}, R^{10b}, and R^{10c} is at least 3, 5, 10, 20, 40, 60, 80, 90, 95, 98, or 99 mol %;
when R³ is

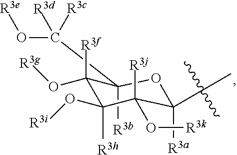

the abundance of deuterium in at least one of R^{1a}, R^{1b}, R^{1c}, R^{2a}, R^{2b}, R^{2c}, R^{3a}, R^{3b}, R^{3c}, R^{3d}, R^{3e}, R^{3f}, R^{3g}, R^{3h}, R³ⁱ, R^{3j}, R^{3k}, R^{7a}, R^{7b}, R^{7c}, R^{10a}, R^{10b}, and R^{10c} is at least 3, 5, 10, 20, 40, 60, 80, 90, 95, 98, or 99 mol %; and
when R³ is

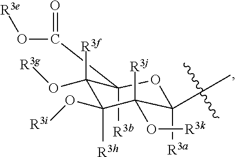

the abundance of deuterium in at least one of R^{1a}, R^{1b}, R^{1c}, R^{2a}, R^{2b}, R^{2c}, R^{3a}, R^{3b}, R^{3c}, R^{3g}, R^{3h}, R³ⁱ, R^{3j}, R^{3k}, R^{7a}, R^{7b}, R^{7c}, R^{10a}, R^{10b}, and R^{10c} is at least 3, 5, 10, 20, 40, 60, 80, 90, 95, 98 or 99 mol %.

In another embodiment, the deuterium-enriched compound is a compound of Formula II,

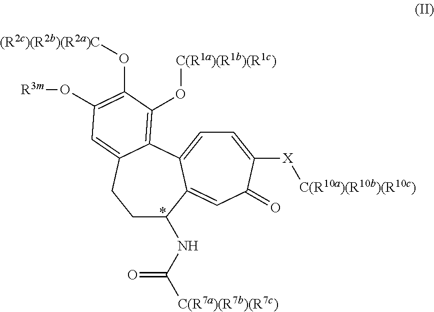

or a pharmaceutically acceptable salt, hydrate, or solvate thereof, wherein

### X is —O— or —S—;

R^{1a}, R^{1b}, R^{1c}, R^{2a}, R^{2b}, R^{2c}, R^{3m}, R^{7a}, R^{7b}, R^{7c}, R^{10a}, R^{10b}, and R^{10c}, are each independently hydrogen or deuterium, provided that the abundance of deuterium in at least one of R^{1a}, R^{1b}, R^{1c}, R^{2a}, R^{2b}, R^{2c}, R^{3m}, R^{7a}, R^{7b}, R^{7c}, R^{10a}, R^{10b}, and R^{10c} is at least 3, 5, 10, 20, 40, 60, 80, 90, 95, 98, or 99 mol %; and
the stereocenter of Formula II indicated by “*” can be racemic, a mixture of R and S enriched in either the R or S isomer, in the R configuration, or in the S configuration.

In yet another embodiment, the deuterium-enriched compound is a compound of Formula III,

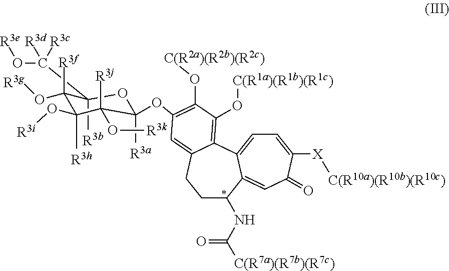

or a pharmaceutically acceptable salt, hydrate, or solvate thereof, wherein:

### X is —O— or —S—;

R^{1a}, R^{1b}, R^{1c}, R^{2a}, R^{2b}, R^{2c}, R^{3a}, R^{3b}, R^{3c}, R^{3d}, R^{3e}, R^{3f}, R^{3g}, R^{3h}, R³ⁱ, R^{3j}, R^{3k}, R^{7a}, R^{7b}, R^{7c}, R^{10a}, R^{10b}, and R^{10c}, are each independently hydrogen or deuterium, provided that the abundance of deuterium in at least one of R^{1a}, R^{1b}, R^{1c}, R^{2a}, R^{2b}, R^{2c}, R^{3a}, R^{3b}, R^{3c}, R^{3d}, R^{3e}, R^{3f}, R^{3g}, R^{3h}, R³ⁱ, R^{3j}, R^{3k}, R^{7a}, R^{7b}, R^{7c}, R^{10a}, R^{10b}, and R^{10c} is at least 3, 5, 10, 20, 40, 60, 80, 90, 95, 98, or 99 mol %; and
the stereocenter of Formula III indicated by “*” can be racemic, a mixture of R and S enriched in either the R or S isomer, in the R configuration, or in the S configuration.
provided that

at least one of R¹, R², R⁷, and R¹⁰ is —CH₂D, —CHD₂, or —CD₃; or
at least one of R^{3a}, R^{3b}, R^{3c}, R^{3d}, R^{3e}, R^{3f}, R^{3g}, R^{3h}, R³ⁱ, R^{3j}, R^{3k} is deuterium.

In still yet another embodiment, the deuterium-enriched compound is a compound of Formula IV,

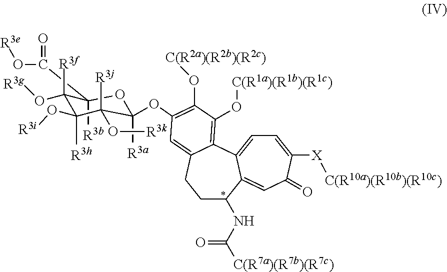

or a pharmaceutically acceptable salt, hydrate, or solvate thereof, wherein:

### X is —O— or —S—;

R^{1a}, R^{1b}, R^{1c}, R^{2a}, R^{2b}, R^{2c}, R^{3a}, R^{3b}, R^{3e}, R^{3f}, R^{3g}, R^{3h}, R³ⁱ, R^{3j}, R^{3k}, R^{7a}, R^{7b}, R^{7c}, R^{10a}, R^{10b}, and R^{10c} are each independently hydrogen or deuterium, provided that the abundance of deuterium in at least one of R^{1a}, R^{1b}, R^{1c}, R^{2a}, R^{2b}, R^{2c}, R^{3a}, R^{3b}, R^{1f}, R^{3g}, R^{3h}, R³ⁱ, R^{3j}, R^{3k}, R^{7a}, R^{7b}, R^{7c} R^{10a}, R^{10b}, and R^{10c} is at least 3, 5, 10, 20, 40, 60, 80, 90, 95, 98, or 99 mol %; and
the stereocenter of Formula IV indicated by “*” can be racemic, a mixture of R and S enriched in either the R or S isomer, in the R configuration, or in the S configuration.

In an additional embodiment, the deuterium-enriched compound of Formula I, II, III, or IV comprises an abundance of deuterium in at least one of R^{1a}, R^{1b}, and R^{1c} of at least 3, 5, 10, 20, 40, 60, 80, 90, 95, 98, or 99 mol %.

In an additional embodiment, the deuterium-enriched compound of Formula I, II, III, or IV comprises an abundance of deuterium in at least one of R^{2a}, R^{2b}, and R^{2c} of at least 3, 5, 10, 20, 40, 60, 80, 90, 95, 98, or 99 mol %.

In an additional embodiment, the deuterium-enriched compound of Formula I comprises an abundance of deuterium in at least one of R^{3a}, R^{3b}, and R^{3c} of at least 3, 5, 10, 20, 40, 60, 80, 90, 95, 98, or 99 mol %.

In one embodiment, the deuterium-enriched compound of Formula I, II, III, or IV comprises an abundance of deuterium in at least one of R^{7a}, R^{7b}, and R^{7c} of at least 3, 5, 10, 20, 40, 60, 80, 90, 95, 98, or 99 mol %.

In another embodiment, the deuterium-enriched compound of Formula I, II, III, or IV comprises an abundance of deuterium in at least one of R^{10a}, R^{10b}, and R^{10c} of at least 3, 5, 10, 20, 40, 60, 80, 90, 95, 98, or 99 mol %.

In another embodiment, the deuterium-enriched compound of Formula II comprises an abundance of deuterium in at least one of R^{1a}, R^{1b}, and R^{1c} of at least 50 mol %.

In another embodiment, the deuterium-enriched compound of Formula II comprises an abundance of deuterium in R^{1a}, R^{1b}, and R^{1c} of at least 50 mol %.

In another embodiment, the deuterium-enriched compound of Formula II comprises an abundance of deuterium in at least one of R^{2a}, R^{2b}, R^{2c} of at least 50 mol %.

In another embodiment, the deuterium-enriched compound of Formula II comprises an abundance of deuterium in R^{2a}, R^{2b}, R^{2c} of at least 50 mol %.

In another embodiment, the deuterium-enriched compound of Formula II comprises an abundance of deuterium in at least one of R^{7a}, R^{7b}, and R^{7c} of at least 50 mol %.

In another embodiment, the deuterium-enriched compound of Formula II comprises an abundance of deuterium in R^{7a}, R^{7b}, and R^{7c} of at least 50 mol %.

In another embodiment, the deuterium-enriched compound of Formula II comprises an abundance of deuterium in at least one of R^{10a}, R^{10b}, and R^{10c} of at least 50 mol %.

In another embodiment, the deuterium-enriched compound of Formula II comprises an abundance of deuterium in R^{10a}, R^{10b}, and R^{10c} is at least 50 mol %.

In another embodiment, the deuterium-enriched compound of Formula III comprises an abundance of deuterium in at least one of R^{1a}, R^{1b}, and R^{1c} of at least 50 mol %.

In another embodiment, the deuterium-enriched compound of Formula III comprises an abundance of deuterium in R^{1a}, R^{1b}, and R^{1c} of at least 50 mol %.

In another embodiment, the deuterium-enriched compound of Formula III comprises an abundance of deuterium in at least one of R^{2a}, R^{2b}, R^{2c} of at least 50 mol %.

In another embodiment, the deuterium-enriched compound of Formula III comprises an abundance of deuterium in R^{2a}, R^{2b}, R^{2c} of at least 50 mol %.

In another embodiment, the deuterium-enriched compound of Formula III comprises an abundance of deuterium in at least one of R^{7a}, R^{7b}, and R^{7c} of at least 50 mol %.

In another embodiment, the deuterium-enriched compound of Formula III comprises an abundance of deuterium in R^{7a}, R^{7b}, and R^{7c} of at least 50 mol %.

In another embodiment, the deuterium-enriched compound of Formula III comprises an abundance of deuterium in at least one of R^{10a}, R^{10b}, and R^{10c} of at least 50 mol %.

In another embodiment, the deuterium-enriched compound of Formula III comprises an abundance of deuterium in R^{10a}, R^{10b}, and R^{10c} of at least 50 mol %.

In another embodiment, the deuterium-enriched compound of Formula III comprises an abundance of deuterium in at least one of R^{3a}, R^{3b}, R^{3c}, R^{3d}, R^{3f}, R^{3h}, and R^{3j} of at least 50 mol %.

In another embodiment, the deuterium-enriched compound of Formula III comprises an abundance of deuterium in R^{3a}, R^{3b}, R^{3c}, R^{3d}, R^{3f}, R^{3h}, and R^{3j} of at least 50 mol %.

In another embodiment, the deuterium-enriched compound of Formula IV comprises an abundance of deuterium in at least one of R^{1a}, R^{1b}, and R^{1c} of at least 50 mol %.

In another embodiment, the deuterium-enriched compound of Formula IV comprises an abundance of deuterium in R^{1a}, R^{1b}, and R^{1c} of at least 50 mol %.

In another embodiment, the deuterium-enriched compound of Formula IV comprises an abundance of deuterium in at least one of R^{2a}, R^{2b}, and R^{2c} of at least 50 mol %.

In another embodiment, the deuterium-enriched compound of Formula IV comprises an abundance of deuterium in R^{2a}, R^{2b}, and R^{2c} of at least 50 mol %.

In another embodiment, the deuterium-enriched compound of Formula IV comprises an abundance of deuterium in at least one of R^{7a}, R^{7b}, and R^{7c} of at least 50 mol %.

In another embodiment, the deuterium-enriched compound of Formula IV comprises an abundance of deuterium in R^{7a}, R^{7b}, and R^{7c} of at least 50 mol %.

In another embodiment, the deuterium-enriched compound of Formula IV comprises an abundance of deuterium in at least one of R^{10a}, R^{10b}, and R^{10c} of at least 50 mol %.

In another embodiment, the deuterium-enriched compound of Formula IV comprises an abundance of deuterium in R^{10a}, R^{10b}, and R^{10c} of at least 50 mol %.

In another embodiment, the deuterium-enriched compound of Formula IV comprises an abundance of deuterium in at least one of R^{3a}, R^{3b}, R^{3f}, R^{3h}, and R^{3j} of at least 50 mol %.

In another embodiment, the deuterium-enriched compound of Formula IV comprises an abundance of deuterium in R^{3a}, R^{3b}, R^{3f}, R^{3h}, and R^{3j} of at least 50 mol %.

In one embodiment, the deuterium-enriched compound of Formula I, II, III, or IV has X is —S—.

In one embodiment, the deuterium-enriched compound of Formula I, II, III, or IV has the stereocenter indicated by “*” in the S configuration.

Compounds are described using standard nomenclature. For example, any position not substituted by any indicated group is understood to have its valency filled by a bond as indicated, or a hydrogen atom.

The deuterium-enriched compounds of Formulas I, II, III, and IV can be prepared using standard techniques and processes known in the art. Forming a deuterium-enriched compound can be achieved by exchanging protons with deuterium using an enriched solvent (e.g., D₂O or MeOD), by synthesizing the compound with enriched starting materials, or a combination thereof. Exemplary starting materials include deuterium-enriched alkylating reagents, deuterium-enriched acylating reagents, and the like and other enriched starting materials, many of which are commercially available (e.g., methyl-d₃ trifluoromethane sulfonate CAS No. 73900-07-9; acetic anhydride-d₆ CAS No. 16649-49-3; acetic acid-2,2,2-d₃ CAS No. 1112-02-3; D-glucose-1,2,3,4,5,6,6-d₇ CAS No. 23403-54-5; D-glucose-2-d₁ CAS No. 30737-83-8; D-glucose-3-d₁; D-glucose-6,6-d₂ CAS No. 18991-62-3; D-glucose-d₁₂; and methane-d₃-thiol; each available from Sigma-Aldrich). Appropriate deuterium-enriched reagents can also be prepared by a supplier specializing in custom synthesis of labeled compounds, such as Cambridge Isotope Laboratories, Inc. Andover, Mass.; SRI International, Menlo Park, Calif.; ChemSyn Laboratories, Lexena, Kans.; American Radiolabeled Chemicals, Inc., St. Louis, Mo.; and Moravek Biochemicals Inc., Brea, Calif.

The extent of deuterium exchange or enrichment can be determined using techniques well known in the art including mass spectrometry and nuclear magnetic resonance spectroscopy.

Starting materials for the preparation of the deuterium-enriched compounds of Formulas I, II, III, and IV include colchicine, thiocolchicine, 3-O-demethylthiocolchicine (CAS No. 87424-25-7), 3-O-demethyl-N-deacetylcolchicine, 3-O-demethyl-N-deacetylthiocolchicine, N-deacetylcolchicine, N-deacetylthiocolchicine, and the like. Processes to obtain such materials can be found in U.S. Pat. Nos. 4,692,463 (2,3-O-didemethylcolchicine and 2,3-O— didemethylthiocolchicine); 5,175,342; 5,880,160; and 6,080,739. An exemplary glucosylation reaction can be found in U.S. Pat. No. 5,777,136 wherein a corresponding deuterated version of glucose or appropriately protected (acetylated) derivative thereof may be used.

U.S. Pat. No. 2,820,029 discloses the reaction of methylmercaptan with colchicine to form thiocolchicine. Replacement of methylmercaptan with methane-d₃-thiol will afford the deuterated analog.

Other processes to prepare the deuterium-enriched compounds are well within the knowledge of a skilled artisan.

An “active agent” means a compound, element, or mixture that when administered to a patient, alone or in combination with another compound, element, or mixture, confers, directly or indirectly, a physiological effect on the patient. The indirect physiological effect may occur via a metabolite or other indirect mechanism. When the active agent is a compound, then salts, solvates (including hydrates) of the free compound or salt, crystalline forms, non-crystalline forms (amorphous), and any polymorphs of the compound are contemplated herein. Compounds may contain one or more asymmetric elements such as stereogenic centers, stereogenic axes and the like, e.g., asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, all optical isomers in pure form and mixtures thereof are encompassed. In addition, compounds with carbon-carbon double bonds may occur in Z- and E-forms, with all isomeric forms of the compounds. In these situations, the single enantiomers, i.e., optically active forms can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column. All forms are contemplated herein regardless of the methods used to obtain them.

“Pharmaceutically acceptable salts” includes derivatives of an active agent, wherein the active agent is modified by making acid or base addition salts thereof, and further refers to pharmaceutically acceptable solvates, including hydrates, crystalline forms, non-crystalline forms, polymorphs, and stereoisomers of such salts. Examples of pharmaceutically acceptable salts include, but are not limited to, mineral or organic acid addition salts of basic residues such as amines; alkali or organic addition salts of acidic residues; and the like, or a combination comprising one or more of the foregoing salts. The pharmaceutically acceptable salts include salts and the quaternary ammonium salts of the active agent. For example, acid salts include those derived from inorganic acids such as hydrochloric, hydrobromic, sulfuric, sulfamic, phosphoric, nitric and the like; other acceptable inorganic salts include metal salts such as sodium salt, potassium salt, cesium salt, and the like; and alkaline earth metal salts, such as calcium salt, magnesium salt, and the like, or a combination comprising one or more of the foregoing salts. Pharmaceutically acceptable organic salts includes salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like; organic amine salts such as triethylamine salt, pyridine salt, picoline salt, ethanolamine salt, triethanolamine salt, dicyclohexylamine salt, N,N′-dibenzylethylenediamine salt, and the like; and amino acid salts such as arginate, asparginate, glutamate, and the like; or a combination comprising one or more of the foregoing salts.

The deuterium-enriched compound can be formulated for oral, buccal, sublingual, mucosal, transdermal, rectal, vaginal, subcutaneous, intramuscular, and intravenous delivery.

In one embodiment, a pharmaceutical formulation comprises a deuterium-enriched compound of Formula I, II, III, or IV.

By “oral dosage form” is meant to include a unit dosage form of the active agent for oral administration that may be solid, semisolid, or liquid. An oral dosage form may optionally comprise a plurality of subunits such as, for example, microcapsules or microtablets. Multiple subunits may be packaged for administration in a single dose. Other exemplary dosage forms for oral administration include, for example, suspension, an emulsion, an orally disintegrating tablet including an effervescent tablet, a sublingual tablet, an orally dissolving strip, a gastro-resistant tablet, a soft capsule, a hard capsule, a gastro-resistant capsule, a tablet, a coated granule, a gastro-resistant granule, and the like.

By “subunit” is meant to include a composition, mixture, particle, pellet, etc., that can provide an oral dosage form alone or when combined with other subunits.

Solid dosage forms for oral administration include, but are not limited to, capsules, tablets, powders, and granules. In such solid dosage forms, the active agent may be admixed with one or more of the following: (a) one or more inert excipients (or carriers), such as sodium citrate or dicalcium phosphate; (b) fillers or extenders, such as starches, lactose, sucrose, glucose, mannitol, and silicic acid; (c) binders, such as carboxymethylcellulose, alignates, gelatin, polyvinylpyrrolidone, sucrose, and acacia; (d) humectants, such as glycerol; (e) disintegrating agents, such as agar-agar, calcium carbonate, potato or tapioca starch, alginic acid, certain complex silicates, and sodium carbonate; (1 solution retarders, such as paraffin; (g) absorption accelerators, such as quaternary ammonium compounds; (h) wetting agents, such as cetyl alcohol and glycerol monostearate; (i) adsorbents, such as kaolin and bentonite; and (j) lubricants, such as talc, calcium stearate, magnesium stearate, solid polyethylene glycols, sodium lauryl sulfate, or a combination comprising one or more of the foregoing additives. For capsules and tablets, the dosage forms may also comprise buffering agents.

Another suitable oral dosage form is a non-chewable, orally disintegrating tablet. These dosage forms can be made by methods known to those of ordinary skill in the art of pharmaceutical formulations. For example, Cima Labs has produced oral dosage forms including microparticles and effervescents, which rapidly disintegrate in the mouth and provide adequate taste-masking. Cima Labs has also produced a rapidly dissolving dosage form containing the active agent and a matrix that includes a nondirect compression filler and a lubricant. U.S. Pat. No. 5,178,878 and U.S. Pat. No. 6,221,392 provide teachings regarding orally disintegrating tablets.

An exemplary orally disintegrating tablet includes a mixture incorporating a water or saliva activated effervescent disintegration agent and subunits such as coated particles, specifically of a size such that chewing does not damage the structure of the subunit. The mixture including the subunits and effervescent disintegration agent may be formulated as a tablet of a size and shape adapted for direct oral administration to a patient. The tablet is substantially completely disintegrable upon exposure to water or saliva. The effervescent disintegration agent is present in an amount effective to aid in disintegration of the tablet, and to provide a distinct sensation of effervescence when the tablet is placed in the mouth of a patient.

The effervescent sensation is not only pleasant to the patient but also tends to stimulate saliva production, thereby providing additional water to aid in further effervescent action. Thus, once the tablet is placed in the patient's mouth, it will disintegrate rapidly and substantially completely without any voluntary action by the patient. Even if the patient does not chew the tablet, disintegration will proceed rapidly. Upon disintegration of the tablet, the subunits are released and can be swallowed as a slurry or suspension. The subunits thus may be transferred to the patient's stomach for dissolution in the digestive tract and systemic distribution of the active agent.

The term effervescent disintegration agent includes compounds which evolve gas. The preferred effervescent disintegration agents evolve gas by means of chemical reactions which take place upon exposure of the effervescent disintegration agent to water or to saliva in the mouth. The bubble or gas generating reaction is most often the result of the reaction of a soluble acid source and an alkali metal carbonate or carbonate source. The reaction of these two general classes of compounds produces carbon dioxide gas upon contact with water included in saliva.

Such water activated materials may be kept in a generally anhydrous state with little or no absorbed moisture or in a stable hydrated form since exposure to water will prematurely disintegrate the tablet. The acid sources or acid may be those which are safe for human consumption and may generally include food acids, acid anhydrides and acid salts. Food acids include citric acid, tartaric acid, malic acid, fumaric acid, adipic acid, and succinic acids etc. Because these acids are directly ingested, their overall solubility in water is less important than it would be if the effervescent tablet formulations were intended to be dissolved in a glass of water. Acid anhydrides and acid of the above described acids may also be used. Acid salts may include sodium, dihydrogen phosphate, disodium dihydrogen pyrophosphate, acid citrate salts and sodium acid sulfite.

Carbonate sources include dry solid carbonate and bicarbonate salts such as sodium bicarbonate, sodium carbonate, potassium bicarbonate and potassium carbonate, magnesium carbonate and sodium sesquicarbonate, sodium glycine carbonate, L-lysine carbonate, arginine carbonate, amorphous calcium carbonate, or a combination comprising at least one of the foregoing carbonates.

The effervescent disintegration agent is not always based upon a reaction which forms carbon dioxide. Reactants which evolve oxygen or other gasses which are safe are also considered within the scope. Where the effervescent agent includes two mutually reactive components, such as an acid source and a carbonate source, it is preferred that both components react substantially completely. Therefore, an equivalent ratio of components which provides for equal equivalents is preferred. For example, if the acid used is diprotic, then either twice the amount of a mono-reactive carbonate base, or an equal amount of a di-reactive base should be used for complete neutralization to be realized. However, the amount of either acid or carbonate source may exceed the amount of the other component. This may be useful to enhance taste or performance of a tablet containing an overage of either component. In this case, it is acceptable that the additional amount of either component may remain unreacted.

In general, the amount of effervescent disintegration agent useful for the formation of tablets is about 5 wt % to about 50 wt % based on the total weight of the final dosage form, specifically about 15 wt % and about 30 wt %, and more specifically about 20 wt % to about 25 wt %.

Other types of orally disintegrating tablets can be prepared without an effervescent agent by using a spray dried carbohydrate or sugar alcohol excipients (e.g. sorbitol, mannitol, xylitol, or a combination comprising at least one of the foregoing, and the like), optionally combined with a disintegrant (e.g. the disintegrant is selected from crospovidone, croscarmellose, sodium starch glycolate, pregelatinized starch, partially pregelatinized starch, or a combination comprising at least one of the foregoing, and the like), or a glidant (e.g. colloidal silica, silica gel, precipitated silica, or a combination comprising at least one of the foregoing, and the like). Suitable orally disintegrating tablets can be found in U.S. Patent Application Publication US20030118642 A1 to Norman et al. incorporated herein in its entirety.

Orally disintegrating tablets can be manufactured by well-known tableting procedures. In common tableting processes, the material which is to be tableted is deposited into a cavity, and one or more punch members are then advanced into the cavity and brought into intimate contact with the material to be pressed, whereupon compressive force is applied. The material is thus forced into conformity with the shape of the punches and the cavity.

The orally disintegrating tablets typically rapidly disintegrate when orally administered. By “rapid”, it is understood that the tablets disintegrate in the mouth of a patient in less than about 7 minutes, and specifically between about 30 seconds and about 5 minutes, specifically the tablet should dissolve in the mouth between about 45 seconds and about 2 minutes. Disintegration time in the mouth can be measured by observing the disintegration time of the tablet in water at about 37° C. The tablet is immersed in the water without forcible agitation. The disintegration time is the time from immersion for substantially complete dispersion of the tablet as determined by visual observation. As used herein, the term “complete disintegration” of the tablet does not require dissolution or disintegration of the subunits or other discrete inclusions. In one embodiment, disintegration can be determined by USP 32 (Test <701>).

In another embodiment, the orally disintegrating tablets include those having a dissolution rate of more than 65% release of active agent within 15 minutes. A dissolution profile is a plot of the cumulative amount of active agent released as a function of time. A dissolution profile can be measured, for example, utilizing the standard test for dissolution according to USP 32 (Test <711>) or Drug Release Test <724>. A profile is characterized by the test conditions selected such as, for example, apparatus type, shaft speed, temperature, volume, and pH of the dissolution medium. More than one dissolution profile may be measured. For example, a first dissolution profile can be measured at a pH level approximating that of the stomach, and a second dissolution profile can be measured at a pH level approximating that of one point in the intestine or several pH levels approximating multiple points in the intestine.

A highly acidic pH may be employed to simulate the stomach and a less acidic to basic pH may be employed to simulate the intestine. By the term “highly acidic pH” is meant a pH of about 1 to about 4. A pH of about 1.2, for example, can be used to simulate the pH of the stomach. By the term “less acidic to basic pH” is meant a pH of greater than about 4 to about 7.5, specifically about 6 to about 7.5. A pH of about 6 to about 7.5, specifically about 6.8, can be used to simulate the pH of the intestine.

In another embodiment, the deuterium-enriched compound is formulated into an orally dissolving strip, which rapidly dissolves in the mouth to release the active agent within the strip. The orally dissolving strips generally comprise a water soluble polymer and a deuterium-enriched compound. Exemplary classes of water soluble polymers include water soluble cellulosic polymers, water soluble synthetic polymers, water soluble natural gums and polymers or derivatives thereof, or a combination comprising at least one of the foregoing. Exemplary water soluble cellulosic polymers include hydroxypropyl cellulose, hydroxypropylmethyl cellulose, hydroxyethyl cellulose, carboxymethyl cellulose, or a combination comprising at least one of the foregoing. Exemplary water soluble natural gums and polymers include amylose, dextran, casein, pullulan, gelatin, pectin, agar, carrageenan, xanthan gum, tragacanth, guar gum, acacia gum, arabic gum, sodium alginate, zein, or a combination comprising at least one of the foregoing. Exemplary water soluble synthetic polymers include polyethylene glycol, polyethylene oxide, polyvinyl pyrrolidone, polyvinyl alcohol, carboxyvinyl polymers, water soluble polyacrylic acid/acrylate, or a combination comprising at least one of the foregoing.

The water soluble polymer may be present in amounts of about 20 to about 95, specifically about 30 to about 85, and more specifically about 40 to about 75 wt % based on the total weight of the orally dissolving strip.

The orally dissolving strip can further optionally comprise a plasticizer in addition to the water soluble polymer and active agent. Exemplary plasticizers include propylene glycol, glycerin, glycerol, monoacetin, diacetin, triacetin, dimethyl phthalate, diethyl phthalate, dibutyl phthalate, dibutyl sebacate, triethyl titrate, tributyl citrate, triethyl citrate, triethyl acetyl citrate, castor oil, acetylated monoglycerides, sorbitol, or a combination comprising at least one of the foregoing. The plasticizer may be present in amounts of about 0 to about 20, specifically about 1 to about 15, and more specifically about 5 to about 10 wt % based on the total weight of the orally dissolving strip.

The orally dissolving strip can further optionally comprise an emulsifying agent in addition to the water soluble polymer and active agent. Exemplary emulsifying agents include polyvinyl alcohol, a sorbitan esters, a cyclodextrin, benzyl benzoate, glyceryl monostearate, a polyoxyethylene alkyl ether, a polyoxyethylene stearate, poloxamer, a polyoxyethylene castor oil derivative, a hydrogenated vegetable oil, a polysorbate, or a combination comprising at least one of the foregoing

The emulsifying agent may be present in amounts of about 0 to about 20, specifically about 1 to about 15, and more specifically about 5 to about 10 wt % based on the total weight of the orally dissolving strip.

The orally dissolving strip can further optionally comprise a flavor or sweetener in addition to the water soluble polymer and active agent. Exemplary sweeteners include sugar, a monosaccharide, an oligosaccharide, aldose, ketose, dextrose, maltose, lactose, glucose, fructose, sucrose, a sugar polyol (e.g., mannitol, xylitol, sorbitol, erythritol, and the like), artificial sweeteners (e.g., acesulfame potassium, sucralose, aspartame, saccharin, sodium saccharin, and the like) or a combination comprising at least one of the foregoing. The sweetener may be present in amounts of about 0 to about 20, specifically about 1 to about 15, and more specifically about 5 to about 10 wt % based on the total weight of the orally dissolving strip.

In some embodiments, the orally dissolving formulations of the present invention may comprise an excipient. Suitable excipients include, but are not limited to, microcrystalline cellulose, colloidal silicon dioxide, talc, starch, or a combination comprising at least one of the foregoing. In some embodiments, the excipient may include talc as anti-adhering agent.

Other optional components that can be used to prepare the orally dissolving strip include a filler/diluent, a surfactant, a disintegrating agent, an antifoaming agent, an antioxidant, a buffering agent, a color, or a combination comprising at least one of the foregoing.

In one embodiment, particles of the deuterium-enriched compound are coated with a taste-masking polymer for greater patient acceptability. Exemplary taste-making polymers include meth/acrylic and meth/acrylate polymers and copolymers such as Eudragit® polymers from Evonik Industries (amino methacrylate copolymer, Eudragit® E PO, E 100, and E 12,5; and methacrylic acid copolymer Type A, B, and C, Eudragit® L 100, S 100, and L 100-55). Other taste-masking polymers include cellulose acetate phthalate, ethyl vinyl phthalate, polyvinyl acetate phthalate, a hydroxy alkyl cellulose phthalate, or a combination comprising at least one of the foregoing.

The taste-masking polymer can be used in an amount of about 1 to about 35 wt % based on the total weight of active agent and taste-masking polymer, specifically about 3 to about 20 wt %, and more specifically about 5 to about 10 wt %.

In one embodiment, the orally dissolving strip exhibits a drug loading of not more than 50% w/w of the film. Exemplary orally dissolving strips will comprise about 0.01 to about 50 mg of active agent per strip. In another embodiment, the orally dissolving strip has a thickness of about 0.1 to about 5.0 millimeters, specifically about 0.3 to about 4.0 and yet more specifically about 0.5 to about 2.5. In another embodiment the orally dissolving strip has a surface area of about 1.0 to about 6.0, specifically about 1.2 to about 4.0 and yet more specifically about 1.5 to about 2.0 square centimeters.

The orally dissolving strip once placed in the oral cavity may dissolve after less than about 60 seconds, specifically less than 30 seconds, and yet more specifically less than about 20 seconds.

A solvent can be used in the process to prepare the orally dissolving strip, including water, ethanol, 1-butanol, 2-butanol, 2-ethoxyethanol, ethyl acetate, methyl acetate, 3-methyl-1-butanol, methylethyl ketone, 2-methyl-1-propanol, isobutyl acetate, isopropyl acetate ethyl ether, tert-butylmethyl ether acetone, or a combination comprising at least one of the foregoing. The solvent is used for processing and then removed to result in the final product.

Methods of preparing orally dissolving strips involve solvent casting and film coating. The active agent is mixed with film-forming excipients and solvents such as water, ethanol, and the like. A thin coating of the mixture is cast on a moving, inert substrate and the coated substrate is moved through a drying oven to evaporate the solvent before die-cutting the dried film into strips. Another method involves hot-melt extrusion, by melting an active agent and excipient polymer blend which is then extruded through a die under molten conditions. The thin film is then cooled to room temperature and die-cut into strips.

The deuterium-enriched compounds disclosed herein are suitable for treating a patient in need thereof, specifically for use as a non-sedating muscle relaxant. The deuterium-enriched compounds can also be used as an anti-inflammatory agent, an anti-gout agent, and anti-cancer agent, or an anti-prolifertive agent. The deuterium-enriched compound is administered in an amount sufficient to provide the desired therapeutic effect (e.g., muscle relaxant activity) to the patient. Amounts can be determined by the skilled artisan using techniques known in the art. Exemplary amounts of deuterium-enriched compound can be about 0.01 to about 50 mg per day, specifically about 1 to about 40 mg per day, more specifically about 4 to about 30 mg per day, and yet more specifically about 8 to about 20 mg per day.

The deuterium-enriched compounds disclosed herein may also be used as standards for bioanalysis.

The invention is further illustrated by the following non-limiting examples.

### EXAMPLES

### Example 1

### 3-O-d₃-thiocolchicine

### 3-O-demethylthiocolchicine is dissolved in an inert solvent and reacted with methyl-d₃ trifluoromethane sulfonate to afford 3-O-d₃-thiocolchicine

### Example 2

### 3-O-d₃-colchicine

3-O-Demethylcolchicine is dissolved in an inert solvent and reacted with methyl-d₃ trifluoromethane sulfonate to afford 3-O-d₃-colchicine.

### Example 3

### 2,3-O-d₆-thiocolchicine

2,3-O-Didemethylthiocolchicine obtained using the process disclosed in U.S. Pat. No. 4,692,463 Example 4 is dissolved in an inert solvent and reacted with methyl-d₆ trifluoromethane sulfonate to afford 2,3-O-d₆-thiocolchicine.

### Example 4

### 2,3-O— d₆-colchicine

2,3-O-d₆-Colchicine can be prepared using the process of Example 3 above and 2,3-O-didemethylcolchicine as obtained using the process disclosed in U.S. Pat. No. 4,692,463 Example 2.

### Example 5

### S-d₃-Thiocolchicine

Colchicine is converted to S-d₃-thiocolchicine using the process disclosed in U.S. Pat. No. 2,820,029 Example 1, using methane-d₃-thiol in place of methyl mercaptan.

### Example 6

### 3-O-demethyl-S-d₃-Thiocolchicine

### 3-O-demethyl-S-d₃-thiocolchicine can be prepared by demethylating S-d₃-thiocolchicine using the process of Example 3 of U.S. Pat. No. 4,692,463.

### Example 7

### acetyl-d₃-thiocolchicine

N-deacetylthiocolchicine is prepared from thiocolchicine using the process disclosed in U.S. Pat. No. 6,080,739. N-deacetylthiocolchicine is reacted with acetic anhydride-d₆ in methylene chloride and triethyl amine to afford d₃-acetyl-thiocolchicine.

N-deacetylthiocolchicine is reacted with acetic acid-2,2,2-d₃ in the presence of a carbodiimide coupling reagent to afford acetyl-d₃-thiocolchicine. Exemplary carbodiimide coupling reagents include dicyclohexylcarbodiimide, diisopropylcarbodiimide, or ethyl-(N′,N′-dimethylamino)propylcarbodiimide hydrochloride. See, Shelkov et al. “Selective esterifications of alcohols and phenols through carbodiimide couplings” Org. Biomol. Chem., 2004, 2, 397-401.

### Example 8

### acetyl-d₃-colchicine

N-deacetylcolchicine is prepared from colchicine using the process disclosed in U.S. Pat. No. 6,080,739. N-deacetylcolchicine is reacted with acetic anhydride-d₆ in methylene chloride and triethyl amine to afford acetyl-d₃-colchicine.

b. N-deacetylcolchicine is reacted with acetic acid-2,2,2-d₃ in the presence of a carbodiimide coupling reagent to afford acetyl-d₃-colchicine.

### Example 9

### 3-O-demethyl-acetyl-d₃-thiocolchicine

### 3-O-demethyl-acetyl-d₃-thiocolchicine can be prepared by demethylating acetyl-d₃-thiocolchicine using the process of Example 3 of U.S. Pat. No. 4,692,463.

### Example 10

### 3-O-demethyl-d₃-acetyl-d₃-colchicine

### 3-O-demethyl-d₃-acetyl-d₃-colchicine can be prepared by demethylating acetyl-d₃-colchicine using the process of Example 1 of U.S. Pat. No. 4,692,463.

### Example 11

### Deuterium-Enriched Thiocolchicoside

Deuterium-enriched thiocolchicoside can be prepared by reacting a deuterium-enriched 2,3,4,6-tetra-O-acetyl-alpha-D-glucopyranosyl fluoride with 3-O-demethylthiocolchicine followed by deprotection using procedures outlined in U.S. Pat. No. 5,777,136. Deuterium-enriched glucose which can be used as starting materials include D-glucose-1,2,3,4,5,6,6-d₇ CAS No. 23403-54-5; D-glucose-2-d₁ CAS No. 30737-83-8; D-glucose-3-d₁; D-glucose-6,6-d₂CAS No. 18991-62-3; and D-glucose-d₁₂ available from Sigma-Aldrich.

### Example 12

### Deuterium-Enriched 3-O-demethylthiocolchicine glucuronate

Deuterium-enriched thiocolchicoside (e.g. from Example 11 above) is converted to 3-O-demethylthiocolchicine glucuronate by an oxidative process using immobilized laccase or Trametes pubescens laccase enzymatic solution and 2,2,6,6-tetramethyl-1-piperidinyloxy (TEMPO) according to Baratto et al. Journal of Molecular Catalysis B: Enzymatic 39 (2006) 3-8.

### Example 13

### Deuterium-Enriched 3-O-demethylcolchicine glucuronate

Deuterium-enriched colchicoside can be prepared from deuterium-enriched colchicoside according to the process in Example 12.

The foregoing examples are representative only. It is well within the purview of the skilled artisan to prepare a variety of deuterium-enriched compounds of any mol % of deuterium. Additional deuterium-enriched compounds of Formula I, II, III, and IV can be prepared using processes and deuterium-enriched reagents well known to the skilled artisan of organic chemistry.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

All cited patents, patent applications, and other references are incorporated herein by reference in their entirety. However, if a term in the present application contradicts or conflicts with a term in the incorporated reference, the term from the present application takes precedence over the conflicting term from the incorporated reference.

All ranges disclosed herein are inclusive of the endpoints, and the endpoints are independently combinable with each other.

The use of the terms “a” and “an” and “the” and similar referents in the context of describing the invention (especially in the context of the following claims) are to be construed to cover both the singular and the plural, unless otherwise indicated herein or clearly contradicted by context. Further, it should further be noted that the terms “first,” “second,” and the like herein do not denote any order, quantity, or importance, but rather are used to distinguish one element from another. The modifier “about” used in connection with a quantity is inclusive of the stated value and has the meaning dictated by the context (e.g., it includes the degree of error associated with measurement of the particular quantity). The term “or” means “and/or” except when describing alternative embodiments of the abundance of mol %, in which case “or” means the alternative only.

1. A deuterium-enriched compound of Formula I, 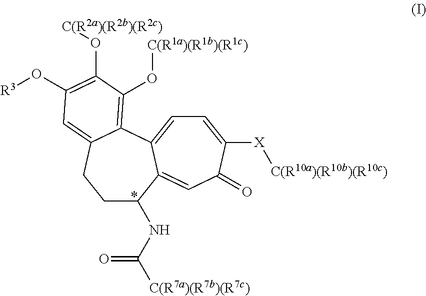
or a pharmaceutically acceptable salt, hydrate, or solvate thereof, wherein:
X is —O— or —S—;
R^{1a}, R^{1b}, R^{1c}, R^{2a}, R^{2b}, R^{2c}, R^{3m}, R^{7a}, R^{7b}, R^{7c}, R^{10a}, R^{10b}, and R^{10c}, are each independently hydrogen or deuterium; and
R³ is
R^{3m}, wherein R^{3m} is hydrogen, deuterium, or C(R^{3a})(R^{3b})(R^{3c}) wherein R^{3a}, R^{3b}, R^{3c}, are each independently hydrogen or deuterium, or 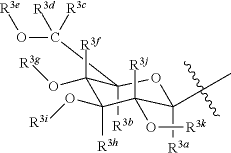
wherein R^{3a}, R^{3b}, R^{3c}, R^{3d}, R^{3e}, R^{3f}, R^{3g}, R^{3h}, R³ⁱ, R^{3j}, R^{3k} are each independently hydrogen or deuterium, or 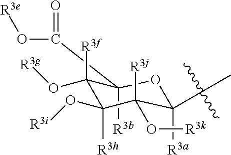
wherein R^{3a}, R^{3b}, R^{3e}, R^{3f}, R^{3g}, R^{3h}, R³ⁱ, R^{3j}, R^{3k} are each independently hydrogen or deuterium; and
the stereocenter of Formula I indicated by “*” can be racemic, a mixture of R and S enriched in either the R or S isomer, in the R configuration, or in the S configuration;
provided that
when R³ is R^{3m}, the abundance of deuterium in at least one of R^{1a}, R^{1b}, R^{1c}, R^{2a}, R^{2b}, R^{2c}, R^{3a}, R^{3b}, R^{3c}, R^{3m}, R^{7a}, R^{7b}, R^{7c}, R^{10a}, R^{10b}, and R^{10c} is at least 3, 5, 10, 20, 40, 60, 80, 90, 95, 98, or 99 mol %;
when R³ is 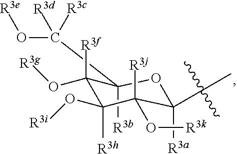
the abundance of deuterium in at least one of R^{1a}, R^{1b}, R^{1c}, R^{2a}, R^{2b}, R^{2c}, R^{3a}, R^{3b}, R^{3c}, R^{3d}, R^{3e}, R^{3f}, R^{3g}, R^{3h}, R³ⁱ, R^{3k}, R^{7a}, R^{7b}, R^{7c}, R^{10a}, R^{10b}, and R^{10c} is at least 3, 5, 10, 20, 40, 60, 80, 90, 95, 98, or 99 mol %; and
when R³ is 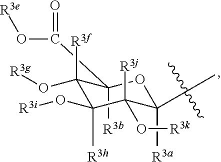
the abundance of deuterium in at least one of R^{1a}, R^{1b}, R^{1c}, R^{2a}, R^{2b}, R^{2c}, R^{3a}, R^{3b}, R^{3c}, R^{3g}, R^{3h}, R³ⁱ, R^{3j}, R^{3k}, R^{7a}, R^{7b}, R^{7c}, R^{10a}, R^{10b}, and R^{10c} is at least 3, 5, 10, 20, 40, 60, 80, 90, 95, 98, or 99 mol %.

2. The deuterium-enriched compound of claim 1, wherein
R³ is C(R^{3a})(R^{3b})(R^{3c}) wherein R^{3a}, R^{3b}, R^{3c} are each independently hydrogen or deuterium; and
X is —O—.

3. The deuterium-enriched compound of claim 1, wherein
R³ is C(R^{3a})(R^{3b})(R^{3c}) wherein R^{3a}, R^{3b}, R^{3c} are each independently hydrogen or deuterium; and
X is —S—.

4. The deuterium-enriched compound of claim 1, wherein the abundance of deuterium in at least one of R^{1a}, R^{1b}, and R^{1c} is at least 3, 5, 10, 20, 40, 60, 80, 90, 95, 98, or 99 mol %.

5. The deuterium-enriched compound of claim 1, wherein the abundance of deuterium in at least one of R^{2a}, R^{2b}, and R^{2c} is at least 3, 5, 10, 20, 40, 60, 80, 90, 95, 98, or 99 mol %.

6. The deuterium-enriched compound of claim 1, wherein the abundance of deuterium in at least one of R^{7a}, R^{7b}, and R^{7c} is at least 3, 5, 10, 20, 40, 60, 80, 90, 95, 98, or 99 mol %.

7. The deuterium-enriched compound of claim 1, wherein the abundance of deuterium in at least one of R^{10a}, R^{10b}, and R^{10c} is at least 3, 5, 10, 20, 40, 60, 80, 90, 95, 98, or 99 mol %.

8. The deuterium-enriched compound of claim 1, wherein R³ is R^{3m} wherein R^{3m} is hydrogen or deuterium, and wherein
the abundance of deuterium in at least one of R^{1a}, R^{1b}, and R^{1c} is at least 50 mol %;
the abundance of deuterium in at least one of R^{2a}, R^{2b}, and R^{2c} is at least 50 mol %;
the abundance of deuterium in at least one of R^{7a}, R^{7b}, and R^{7c} is at least 50 mol %; or
the abundance of deuterium in at least one of R^{10a}, R^{10b}, and R^{10c} is at least 50 mol %.

9. The deuterium-enriched compound of claim 1, wherein R³ is R^{3m} wherein R^{3m} is hydrogen or deuterium, and wherein
the abundance of deuterium in R^{1a}, R^{1b}, and R^{1c} is at least 50 mol %;
the abundance of deuterium in R^{2a}, R^{2b}, and R^{2c} is at least 50 mol %;
the abundance of deuterium in R^{7a}, R^{7b}, and R^{7c} is at least 50 mol %; or
the abundance of deuterium in R^{10a}, R^{10b}, and R^{10c} is at least 50 mol %.

10. The deuterium-enriched compound of claim 1, wherein R³ is R^{3m} wherein R^{3m} is 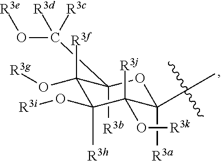
and wherein
the abundance of deuterium in at least one of R^{1a}, R^{1b}, and R^{1c} is at least 50 mol %;
the abundance of deuterium in at least one of R^{2a}, R^{2b}, and R^{2c} is at least 50 mol %;
the abundance of deuterium in at least one of R^{7a}, R^{7b}, and R^{7c} is at least 50 mol %;
the abundance of deuterium in at least one of R^{10a}, R^{10b}, and R^{10c} is at least 50 mol %;
or
the abundance of deuterium in at least one of R^{3a}, R^{3b}, R^{3c}, R^{3d}, R^{3f}, R^{3h}, and R^{3j} is at least 50 mol %.

11. The deuterium-enriched compound of claim 1, wherein R³ is R^{3m} wherein R^{3m} is 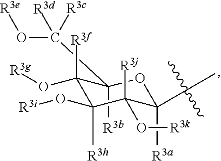
and wherein
the abundance of deuterium in R^{1a}, R^{1b}, and R^{1c} is at least 50 mol %;
the abundance of deuterium in R^{2a}, R^{2b}, and R^{2c} is at least 50 mol %;
the abundance of deuterium in R^{7a}, R^{7b}, and R^{7c} is at least 50 mol %;
the abundance of deuterium in R^{10a}, R^{10b}, and R^{10c} is at least 50 mol %; or
the abundance of deuterium in R^{3a}, R^{3b}, R^{3c}, R^{3d}, R^{3f}, R^{3h}, and R^{3j} is at least 50 mol %.

12. The deuterium-enriched compound of claim 1, wherein R³ is R^{3m} wherein R^{3m} is 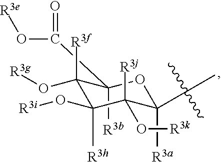
and wherein
the abundance of deuterium in at least one of R^{1a}, R^{1b}, and R^{1c} is at least 50 mol %;
the abundance of deuterium in at least one of R^{2a}, R^{2b}, and R^{2c} is at least 50 mol %;
the abundance of deuterium in at least one of R^{7a}, R^{7b}, and R^{7c} is at least 50 mol %;
the abundance of deuterium in at least one of R^{10a}, R^{10b} and R^{10c} is at least 50 mol %;
or
the abundance of deuterium in at least one of R^{3a}, R^{3b}, R^{3f}, R^{3h} and R^{3j} is at least 50 mol %.

13. The deuterium-enriched compound of claim 1, wherein R³ is R^{3m} wherein R^{3m} is 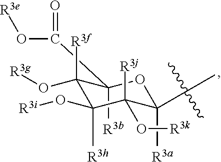
and wherein
the abundance of deuterium in R^{1a}, R^{1b} and R^{1c} is at least 50 mol %;
the abundance of deuterium in R^{2a}, R^{2b}, and R^{2c} is at least 50 mol %;
the abundance of deuterium in R^{7a}, R^{7b}, and R^{7c} is at least 50 mol %;
the abundance of deuterium in R^{10a}, R^{10b}, and R^{10c} is at least 50 mol %; or
the abundance of deuterium in R^{3a}, R^{3b}, R^{3f}, R^{3h}, and R^{3j} is at least 50 mol %.

14. The deuterium-enriched compound of claim 2, wherein
the abundance of deuterium in at least one of R^{1a}, R^{1b}, and R^{1c} is at least 50 mol %;
the abundance of deuterium in at least one of R^{2a}, R^{2b}, and R^{2c} is at least 50 mol %;
the abundance of deuterium in at least one of R^{7a}, R^{7b}, and R^{7c} is at least 50 mol %; or
the abundance of deuterium in at least one of R^{10a}, R^{10b} and R^{10c} is at least 50 mol %.

15. The deuterium-enriched compound of claim 2, wherein
the abundance of deuterium in R^{1a}, R^{1b}, and R^{1c} is at least 50 mol %;
the abundance of deuterium in R^{2a}, R^{2b}, and R^{2c} is at least 50 mol %;
the abundance of deuterium in R^{7a}, R^{7b}, and R^{7c} is at least 50 mol %; or
the abundance of deuterium in R^{10a}, R^{10b} and R^{10c} is at least 50 mol %.

16. The deuterium-enriched compound of claim 3, wherein
the abundance of deuterium in at least one of R^{1a}, R^{1b}, and R^{1c} is at least 50 mol %;
the abundance of deuterium in at least one of R^{2a}, R^{2b}, and R^{2c} is at least 50 mol %;
the abundance of deuterium in at least one of R^{7a}, R^{7b}, and R^{7c} is at least 50 mol %; or
the abundance of deuterium in at least one of R^{10a}, R^{10b}, and R^{10c} is at least 50 mol %.

17. The deuterium-enriched compound of claim 3, wherein
the abundance of deuterium in R^{1a}, R^{1b}, and R^{1c} is at least 50 mol %;
the abundance of deuterium in R^{2a}, R^{2b}, and R^{2c} is at least 50 mol %;
the abundance of deuterium in R^{7a}, R^{7b}, and R^{7c} is at least 50 mol %; or
the abundance of deuterium in R^{10a}, R^{10b}, and R^{10c} is at least 50 mol %.

18. The deuterium-enriched compound of claim 1, wherein the stereocenter indicated by “*” is in the S configuration.

19. A pharmaceutical composition, comprising a deuterium-enriched compound of Formula I and a pharmaceutically acceptable excipient,
wherein the deuterium-enriched compound of Formula I is 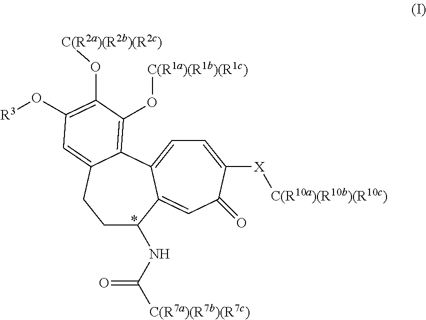
or a pharmaceutically acceptable salt, hydrate, or solvate thereof, wherein:
X is —O— or —S—;
R^{1a}, R^{1b}, R^{1c}, R^{2a}, R^{2b}, R^{2c}, R^{3m}, R^{7a}, R^{7b}, R^{7c}, R^{10a}, R^{10b}, and R^{10c}, are each independently hydrogen or deuterium; and
R³ is
R^{3m}, wherein R^{3m} is hydrogen, deuterium, or C(R^{3a})(R^{3b})(R^{3c}) wherein R^{3a}, R^{3b}, R^{3c}, are each independently hydrogen or deuterium, or 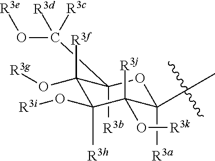
wherein R^{3a}, R^{3b}, R^{3c}, R^{3d}, R^{3e}, R^{3f}, R^{3g}, R^{3h}, R³ⁱ, R^{3j}, R^{3k} are each independently hydrogen or deuterium, or 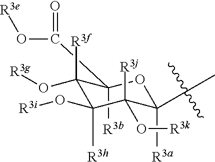
wherein R^{3a}, R^{3b}, R^{3c}, R^{3f}, R^{3g}, R^{3h}, R³ⁱ, R^{3j}, R^{3k} are each independently hydrogen or deuterium; and
the stereocenter of Formula I indicated by “*” can be racemic, a mixture of R and S enriched in either the R or S isomer, in the R configuration, or in the S configuration;
provided that
when R³ is R^{3m}, the abundance of deuterium in at least one of R^{1a}, R^{1b}, R^{1c}, R^{2a}, R^{2b}, R^{2c}, R^{3a}, R^{3b}, R^{3c}, R^{3m}, R^{7a}, R^{7b}, R^{7c}, R^{10a}, R^{10b}, and R^{10c} is at least 3, 5, 10, 20, 40, 60, 80, 90, 95, 98, or 99 mol %;
when R³ is 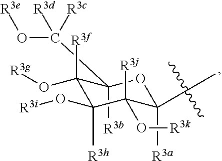
the abundance of deuterium in at least one of R^{1a}, R^{1b}, R^{1c}, R^{2a}, R^{2b}, R^{2c}, R^{3a}, R^{3b}, R^{3c}, R^{3d}, R^{3e}, R^{3f}, R^{3g}, R^{3h}, R³ⁱ, R^{3j}, R^{3k}, R^{7a}, R^{7b}, R^{7c}, R^{10a}, R^{10b}, and R^{10c} is at least 3, 5, 10, 20, 40, 60, 80, 90, 95, 98, or 99 mol %; and
when R³ is 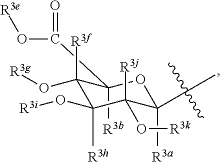
the abundance of deuterium in at least one of R^{1a}, R^{1b}, R^{1c}, R^{2a}, R^{2b}, R^{2c}, R^{3a}, R^{3b}, R^{3f}, R^{3g}, R^{3h}, R³ⁱ, R^{3j}, R^{3k}, R^{7a}, R^{7b}, R^{7c}, R^{10a}, R^{10b}, and R^{10c} is at least 3, 5, 10, 20, 40, 60, 80, 90, 95, 98, or 99 mol %.

20. A method of treating a patient in need of a muscle relaxant, anti-inflammatory or anti-gout agent, comprising administering to the patient a deuterium-enriched compound of Formula I, optionally in combination with a pharmaceutically acceptable excipient,
wherein the deuterium-enriched compound of Formula I is 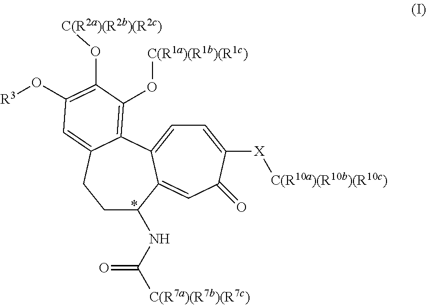
or a pharmaceutically acceptable salt, hydrate, or solvate thereof, wherein:
X is —O— or —S—;
R^{1a}, R^{1b}, R^{1c}, R^{2a}, R^{2b}, R^{2c}, R^{3m}, R^{7a}, R^{7b}, R^{7c}, R^{10a}, R^{10b}, and R^{10c}, are each independently hydrogen or deuterium; and
R³ is
R^{3m}, wherein R^{3m} is hydrogen, deuterium, or C(R^{3a})(R^{3b})(R^{3c}) wherein R^{3a}, R^{3b}, R^{3c}, are each independently hydrogen or deuterium, or 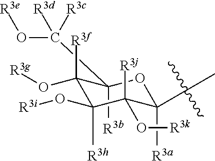
wherein R^{3a}, R^{3b}, R^{3c}, R^{3d}, R^{3e}, R^{3f}, R^{3g}, R^{3h}, R³ⁱ, R^{3j}, R^{3k} are each independently hydrogen or deuterium, or 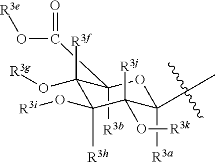
wherein R^{3a}, R^{3b}, R^{3e}, R^{3f}, R^{3g}, R^{3h}, R³ⁱ, R^{3j}, R^{3k} are each independently hydrogen or deuterium; and
the stereocenter of Formula I indicated by “*” can be racemic, a mixture of R and S enriched in either the R or S isomer, in the R configuration, or in the S configuration;
provided that
when R³ is R^{3m}, the abundance of deuterium in at least one of R^{1a}, R^{1b}, R^{1c}, R^{2a}, R^{2b}, R^{2c}, R^{3a}, R^{3b}, R^{3c}, R^{3m}, R^{7a}, R^{7c}, R^{10a}, R^{10b}, and R^{10c} is at least 3, 5, 10, 20, 40, 60, 80, 90, 95, 98, or 99 mol %;
when R³ is 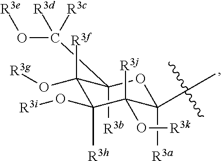
the abundance of deuterium in at least one of R^{1a}, R^{1b}, R^{1c}, R^{2a}, R^{2b}, R^{2c}, R^{3a}, R^{3b}, R^{3c}, R^{3d}, R^{3e}, R^{3f}, R^{3g}, R^{3h}, R³ⁱ, R^{3j}, R^{3k}, R^{7a}, R^{7b}, R^{7c}, R^{10a}, R^{10b}, and R^{10c} is at least 3, 5, 10, 20, 40, 60, 80, 90, 95, 98, or 99 mol %; and
when R³ is 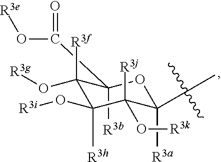
the abundance of deuterium in at least one of R^{1a}, R^{1b}, R^{1c}, R^{2a}, R^{2b}, R^{2c}, R^{3a}, R^{3b}, R^{3f}, R^{3g}, R^{3h}, R³ⁱ, R^{3j}, R^{3k}, R^{7a}, R^{7b}, R^{7c}, R^{10a}, R^{10b}, and R^{10c} is at least 3, 5, 10, 20, 40, 60, 80, 90, 95, 98, or 99 mol %.
